(19) United States
(12) Patent Application Publication
(10) Pub. No.: US 2007/0249902 A1
(43) Pub. Date: Oct. 25, 2007

(54) Dispenser

(75) Inventors: Amram AIZENFELD, Menashe (IL); Golan SALMAN, Tirat Hacarmel (IL); Reuven ZAMIR, Yokneam (IL)

Correspondence Address: BRUCE E. LILLING;LILLING & LILLING PLLC, P.O. BOX 560, GOLDEN BRIDGE, NY, 10526, US

(73) Assignee: STRYKER GI LTD., Haifa (IL)

(21) Appl. No.: 11/692,299

(22) Filed: Mar. 28, 2007

Related U.S. Application Data
(60) Provisional application No. 60/745,496, filed on Apr. 24, 2006.

(51) Int. Cl. A61B 1/00

(52) U.S. Cl. 600/124

(57) ABSTRACT

A dispenser for storing of disposable protecting sleeve is described. The sleeve is intended for covering of at least a portion of a flexible insertion tube of an endoscope. The dispenser comprises frontal housing detachably connectable to a rear housing. The frontal housing comprises cover assembly detachably connected thereto and the rear housing comprises sleeve anchoring member and a retaining assembly. The retaining assembly is provided with a distribution bushing through which a lubricant can be distributed and dislodged on the insertion tube. The distribution bushing is elastically deformable and is deployed within the retaining assembly with possibility for relative rotation with respect to the retaining assembly.

### BACKGROUND OF THE INVENTION

1. Field of the Invention

The present invention relates generally to the field of endoscopy and specifically to endoscopic apparatus used for colonoscopic procedures during which a flexible tube is inserted into the rectum and colon for examination of the colon interior for abnormalities. More particularly, the present invention refers to a dispenser for use with endoscopes having a disposable inflatable sleeve covering the insertion tube and assisting propulsion of the insertion tube within the colon during the endoscopic procedure.

2. Summary of the Prior Art

There are known endoscopes employing inflatable flexible sleeves for propulsion of insertion tubes of an endoscope within the colon.

Voloshin (U.S. Pat. No. 6,485,409) discloses an endoscope, which comprises an endoscopic probe, a bending section for directing the probe within the colon (steering unit), an insertion tube and a flexible covering sleeve or a sheath, which is coupled proximally to the probe. The bending section of the endoscope is located behind the probe. The sleeve is attached to the endoscope in such a manner that its folded section is retained between a cap and an internal spindle, which are located between the insertion tube and the steering unit. When inflated, the folded section unfolds over a flange of the internal spindle and an inner portion of the sleeve is pulled in a distal direction.

Eizenfeld (WO 2004/016299; International application PCT/IL2003/000661), which is herein incorporated by reference, describes an endoscope employing a flexible inflatable sleeve, which before inflation is retained within a dispenser. The dispenser employed in this endoscope has entry and exit ports defining a transit passage through which the endoscope may pass. The dispenser is adapted to capture the sleeve as the endoscope is retracted through the transit passage in a proximal direction. In another embodiment the dispenser includes an external sleeve fixed to the dispenser and this external sleeve is adapted to extend from the dispenser when the endoscope is retracted so that the external sleeve covers the flexible sleeve. By virtue of this provision any contamination on the flexible sleeve remains within the external sleeve and does not contact the endoscope or any other objects or areas outside the patient's body. After the endoscope has been evacuated entirely from the flexible sleeve, the dispenser together with the external sleeve and the flexible sleeve is discarded.

Bar-Or (WO 2005/110185; International application PCT/IL2005/000426), which is herein incorporated by reference, describes a disposable set for use with an endoscopic apparatus. The set comprises a dispenser having a longitudinal transit passage for the insertion tube of the endoscopic apparatus. Construction of the dispenser is designed to allow receiving and distributing of a lubricant within the transit passage before the insertion tube is being passed therealong. The lubricant is distributed in an annular space between the periphery of the insertion tube and a distribution bushing residing in the dispenser. The distribution bushing is fitted with radial openings, which are in fluid communication with the transit passage. The distribution bushing is made of a rigid material and during its manufacturing rather strict dimensional tolerances should be met. Noncompliance with those tolerances might be associated with difficulty or even impossibility in achieving proper sealing between the bushing ends and the adjacent components of the bushing and as a result of this improper functioning of the dispenser and the whole endoscopic apparatus.

Still further disadvantage of the prior art dispenser is associated with the fact that the distribution bushing and the other components of the dispenser are deployed within the dispenser housing in such a manner that the insertion tube passes through the transit passage being displaceable through the dispenser only in the longitudinal direction, without however having a possibility for relative rotational displacement with respect to the housing. This possibility would be desirable if during the colonoscopic procedure the covering sleeve begins to kink and it would be required to prevent or at least to minimize the kinking and entanglement.

### SUMMARY OF THE INVENTION

The main object of the present invention is to provide a new and improved endoscopic apparatus fitted with a disposable inflatable sleeve and with a sleeve-retaining dispenser.

A further object of the invention is to provide a new and improved endoscopic apparatus of the above-mentioned kind, in which reliable sealing between the distribution ring and the adjacent components, as well as reliable anchoring of the covering sleeve in the dispenser, would be always provided after assembling.

Yet another object of the invention is to provide a new and improved endoscopic apparatus of the above-mentioned kind, in which construction of the dispenser provides the possibility for relative rotational movement between the inner components located adjacent with the insertion tube and the external housing of the dispenser.

### BRIEF DESCRIPTION OF THE SEVERAL VIEWS OF THE DRAWINGS

FIG. 1 depicts a general view of a prior art endoscopic apparatus, preferably a colonoscopic apparatus and its main components.

FIG. 2 is a general view of a disposable dispenser and multilumen tubing for use in the endoscopic apparatus shown in FIG. 1.

FIG. 3 depicts the distal end of the multilumen tubing being inserted within the dispenser and its proximal end being ready for insertion within the guide channel of the insertion tube.

FIG. 4 is a general isometric view of the dispenser of the present invention.

FIG. 5a and FIG. 5b show disposable multilumen tubing protruding from the dispenser and being inserted in the guide channel of the insertion tube via optical head.

FIG. 6 is an exploded isometric view of the dispenser of the invention.

FIG. 7 is an enlarged isometric view of a rear assembly used in the dispenser of the invention.

FIG. 8 is an enlarged cross-sectional view of the dispenser of the invention.

FIG. 9 is a cross-sectional view of the rear assembly with the distribution bushing.

FIG. 10a and FIG. 10b show the rear assembly with the distribution bushing, which length is not in compliance with the dimensional tolerances being respectively either too short or too long than the nominal length.

FIG. 11 is an enlarged isometric view of the distribution bushing employed in the dispenser of the invention.

### DETAILED DESCRIPTION OF THE INVENTION

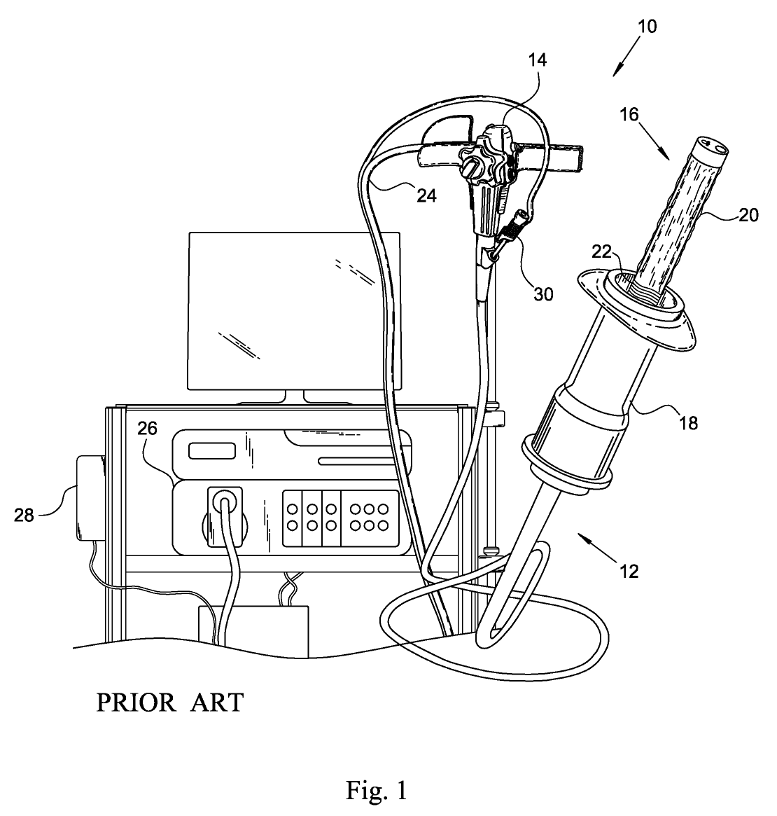

With reference to FIG. 1 the endoscopic apparatus of the present invention is shown preferably as a colonoscopic apparatus 10 with its following main components. The apparatus comprises an endoscope having insertion tube 12 with its proximal section connected to an operation handle 14 and with its distal section 16 inserted in and protruding from a disposable dispenser 18.

It is seen also in FIG. 1 that a sleeve covers distal section 16 of the endoscope. That part of the sleeve, which is seen in FIG. 1, comprises a frontal non-inflatable portion 20 and a rear, folded portion 22. The frontal portion of the sleeve covers the distal section of the endoscope. The frontal portion does not inflate when the endoscope advances within the colon. The rear portion covers the insertion tube and unfolds when air or another fluid medium is supplied to the sleeve. By virtue of this provision the feeding out sleeve assists to propel the insertion tube within the body passage. Explanation of this phenomenon can be found in the above referred-to references.

The endoscope used with the present invention is of similar type in the sense that it employs the same propelling mechanism, which is based on inflation of the flexible disposable sleeve coupled to the endoscope. It should be appreciated, however, that the endoscope of the present invention is not limited merely to colonoscopy. It can be employed in any other medical procedure requiring insertion of a probe in a body passage for inspection of its interior.

It is seen also in FIG. 1, that the handle is connected by appropriate umbilical cord 24 to a control unit 26 provided with a source of compressed air for inflating and venting the sleeve. Proximate to the control unit a flask 28 is provided, which is filled with water, to be supplied under pressure into the colon for irrigation. Water and air are supplied to the handle through dedicated tubes (not shown) attachable to the umbilical duct.

One should bear also in mind that within the insertion tube are provided various devices, which are necessary for proper functioning of the endoscope. These devices are known per se. Among such devices one can mention vertebrae and strings, which can be manipulated by the handle and a multilumen tubing with appropriate passages for supplying water, as required for irrigation or vacuum as required for suction. The multilumen tubing also is provided with a dedicated passage for introducing surgical instruments into the colon as might be required during the endoscopic procedure. Along the insertion tube might extend also a dedicated tube through which air is supplied as required for inflating the sleeve.

The multilumen tubing extends through the endoscope and through the handle to a connector means 30, which provides flow communication between lumens of the multilumen tubing and tubes passing through the umbilical duct and supplying to the insertion tube air and vacuum from the control unit and water from the flask.

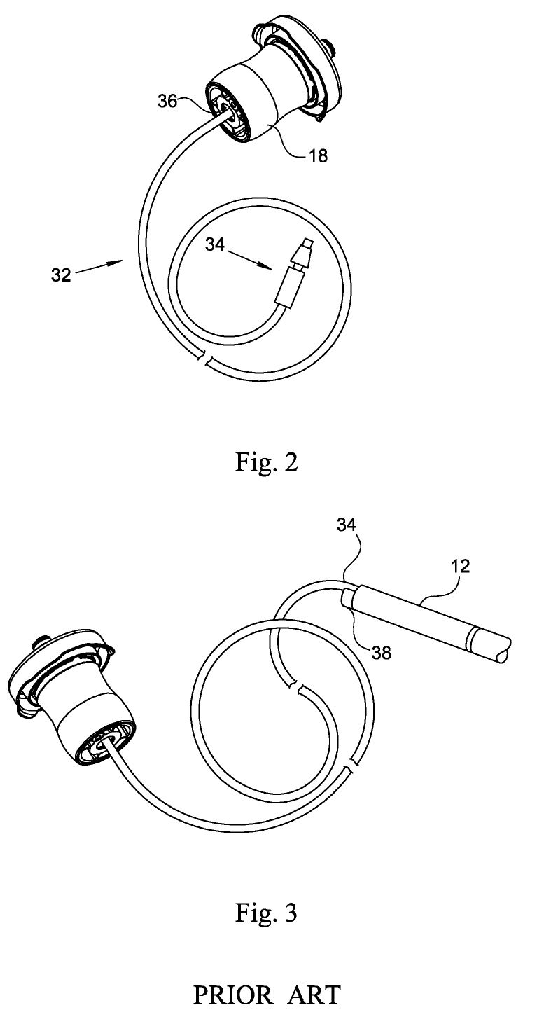

In FIG. 2 is seen a general view of a prior art disposable dispenser 18 and a disposable multilumen tubing 32, having its distal section inserted within the dispenser and its proximal section extending outside the dispenser. An end 34 of the proximal section of the dispenser is adapted for airtight connection to the connector means 30.

It is not shown in FIG. 2, but it should be appreciated, that the distal end of the multilumen tubing is attached to a cap having a window fitted with transparent closure. Through this window illuminating light can propagate and thus viewing optics of a camera head may observe the interior of a body passage when the endoscope is inserted in the body passage.

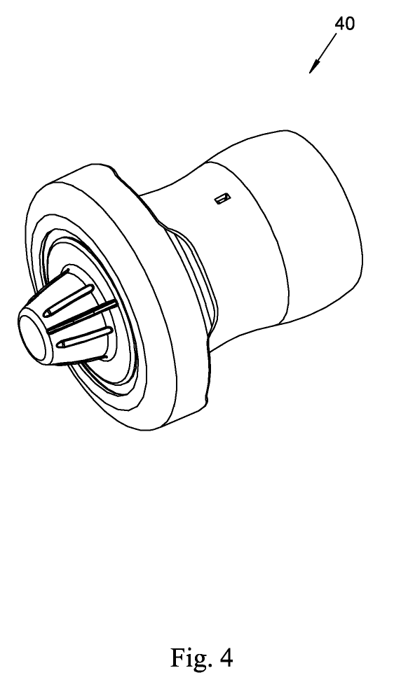

With reference to FIG. 3 it is shown how disposable dispenser and multilumen tubing are used during preparation of the colonoscope to the colonoscopic procedure.

Distal end of the insertion tube 12 is seen with a camera head 38. The distal end is shown before it is introduced within the dispenser and is covered by the cap.

The construction and functioning of the prior art colonoscopic apparatus and the dispenser depicted in FIGS. 1-3 is described in Bar-Or (WO 2005/110185; International application PCT/IL2005/000426).

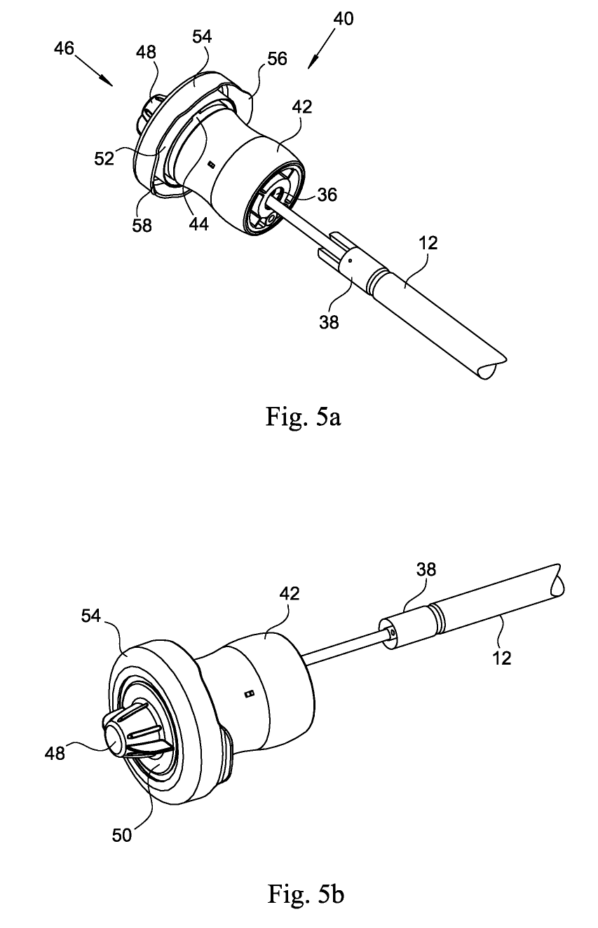

Referring now to FIG. 4 there is shown a dispenser 40 of the present invention, which functionally is similar to the above-mentioned prior art dispenser and which is used during the preparation step in the same manner as can be seen from FIG. 5a and FIG. 5b. In these figures similar reference numbers are used for designating similar elements.

FIGS. 5a and 5b show the multilumen tubing 32 being inserted within a guiding channel of the insertion tube 12 via optical head 38. Then, distal section of the insertion tube is displaced distally and is inserted into dispenser via an entry port 36 in the rear end of the dispenser. After that, the distal section of the insertion tube is advanced distally along the dispenser until it protrudes therefrom. It is seen in FIG. 5b that distal section of the insertion tube terminates by an optical head 38, which has a saddle-like cross-sectional configuration, thereby enabling the multilumen tubing to pass therethrough and through the guiding channel of the insertion tube.

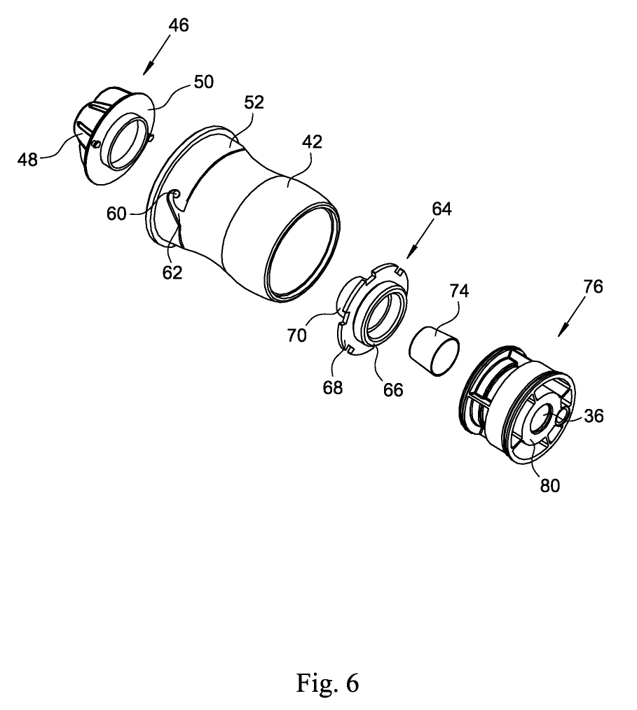

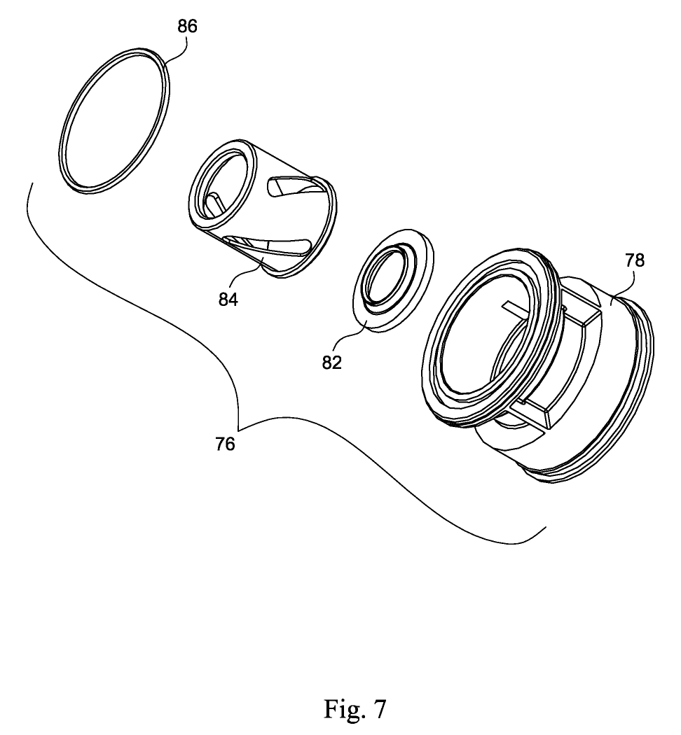

As shown in FIG. 5a and FIG. 5b the dispenser is provided with a rear housing 42 and with removably attached thereto a frontal housing 44. A cover assembly 46 is detachably secured on the frontal housing. The cover assembly has a cover 48 and a flange portion 50. A rear portion 52 of the frontal housing embraces the rear housing. A frontal portion of the frontal housing is provided with a shield means 54 having lateral portions 56, 58. During the endoscopic procedure a dedicated garment (not shown) can support lateral portions of the shield means, for example as explained in Golan (WO 2004/107889; International application PCT/IL2004/000372), which disclosure is herein incorporated by reference. This provision renders the operation of the endoscopic apparatus more convenient. As seen in FIG. 6 a pin 60 protrudes from the periphery of the rear housing for entering into a curved slot 62 made in the rear portion of the frontal housing. The pin and the slot constitute a bayonet and by virtue of this provision, the frontal housing can be attached to or detached from the rear housing by rotation of the frontal housing to engage or disengage the pin from the slot.

With reference to FIGS. 6-8 the other components of the dispenser will be now described.

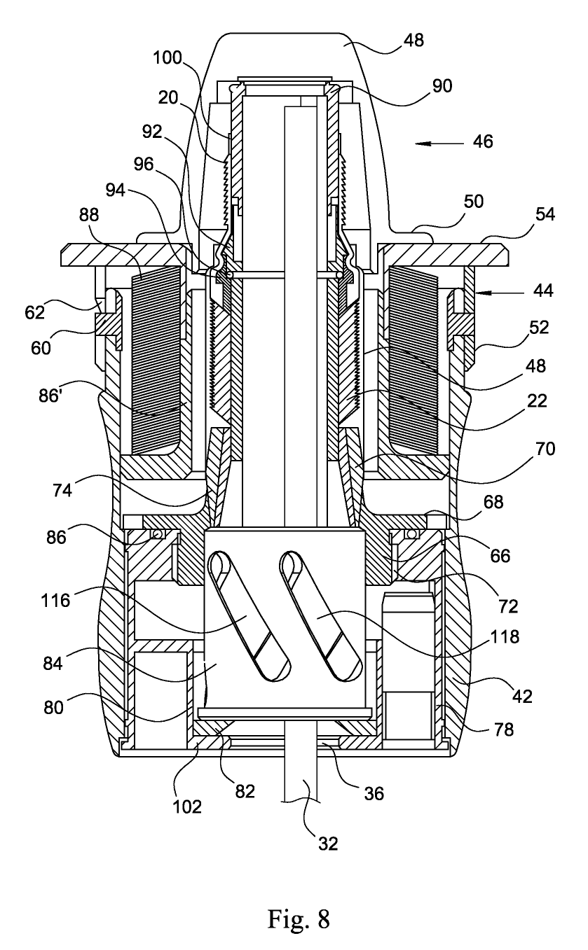

Mounted within the rear housing and shown in FIG. 6 a sleeve-anchoring member 64 is provided. The sleeve-anchoring member has a rear shoulder portion 66, an intermediate flange portion 68 and a frontal skirt portion 70. It is not seen in FIG. 6, but shown in FIG. 8 that the shoulder portion 66 is provided with a thread 72.

Insertable within the skirt portion and tightly sitting therein a conical ring 74 is provided. It will be shown further with reference to FIG. 8 that the rear end of the covering sleeve 22 is anchored between the conical ring and the skirt portion of the sleeve anchoring member.

Mounted within the rear housing of the dispenser a rear retaining assembly 76 is provided, which is configured with a tubular main body portion 78 and with a shoulder 80. As shown in FIG. 7 the main body portion retains therein a proximal sealing element 82, abutted by the shoulder portion. As seen in FIG. 7 within the main body portion is deployed a distribution bushing 84 and a distal sealing element 86. When the dispenser is assembled the proximal sealing element 82 seals between rear end of the distribution bushing 84 and the entry port 36. The distal sealing element 86 comprises an O-ring, which provides sealing between the frontal end of the distribution bushing 84 and the flange portion 68 of the sleeve anchoring member 64.

In FIG. 8 are shown the main components of the dispenser when it is assembled. Except of the already mentioned main components the dispenser is fitted also with a retainer bushing 86′. The retainer bushing is located within the dispenser between the sleeve anchoring member 64 and the shield 54 and it is intended to provide room for an auxiliary sleeve 88. The auxiliary sleeve is used in the final step of the endoscopic procedure when the insertion tube and the covering sleeve is being evacuated from the body channel. This step is explained in Bar-Or (WO 2005/110185; International application PCT/IL2005/000426).

A cap 90 is seen in FIG. 8. At the cap the non-inflatable portion 20 of the covering sleeve is secured. A bushing member 92 and a retainer ring 94 with a sealing ring 96 are also deployed within the frontal housing.

The front end of the unfolded portion 20 is secured at the cap by gluing or by a ring 100 tightly sitting on the outside surface of the cap. The rear end of the folded portion 22 of the sleeve is anchored between conical ring 74 and skirt portion 70 of the sleeve anchoring member 64.

The above-described cap and arrangement for securing the sleeve is described in more detail in Bar-Or (WO 2005/110204; International application PCT/IL2005/000425), which disclosure is herein incorporated by reference.

The dispenser, the multilumen tubing and the tubes extending along the umbilical duct are manufactured from appropriate plastic material; they are cheap, disposable items, which are discarded at the end of the endoscopic procedure after evacuating the insertion tube from the body passage. By virtue of this provision, preparation of the endoscopic apparatus to the new endoscopic procedure is simple, convenient and fast.

The shoulder 80 of the main body portion 78 is provided with a shelf region 102 for abutment the proximal sealing element 82. The sealing element 82 is manufactured from appropriate plastic material, having Shore hardness A, e.g. from silicon rubber.

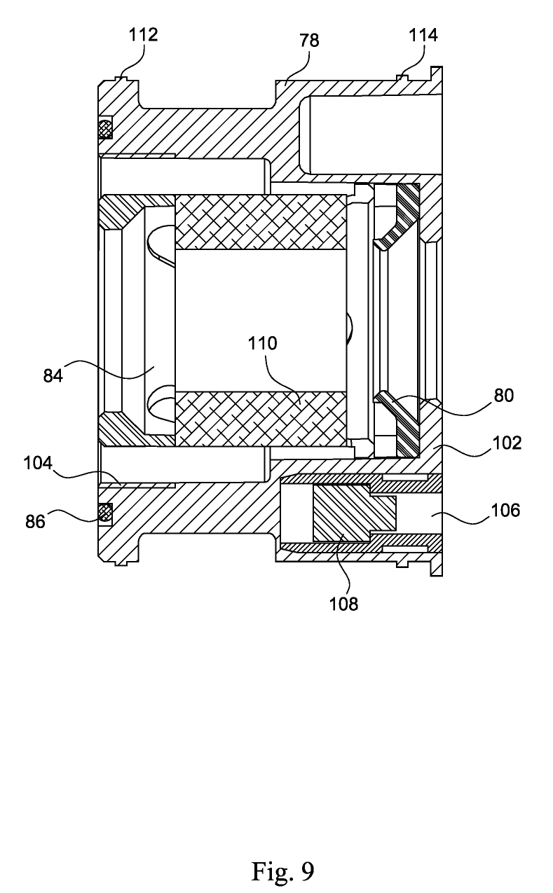

As shown in FIG. 9 the main body portion 78 is provided with a thread 104 for screwing on the shoulder portion 66 of the sleeve-anchoring member 64.

As seen in FIG. 9, the rear end of the main body portion 78 is fitted with a recess 106. A check valve 108 is deployed within the recess and an external syringe can be introduced through the recess for supplying a liquid lubricant through the check valve. A spongy liner 110 is deployed on the inwardly facing surface of the distribution bushing and can be secured thereto for example by gluing. This liner is used for absorbing the lubricant when it enters in the distribution bushing. The absorbed lubricant is dislodged on the insertion tube when it is displaced along the transition path. Similar arrangement is disclosed in more details in Bar-Or (WO 2005/110204; International application PCT/IL2005/000425).

The main body portion 78 is manufactured from rigid plastic material. On the outside periphery of the main body portion there are provided discrete annular protrusions 112, 114. The protrusions function as sliding bearings, which enable relative rotational displacement between the rear housing 42 and the retaining assembly 76. In practice the length of the distribution bushing is about 30 mm, the height of the protrusions is about 0.5 mm and their width is about 1 mm.

Since relative rotational displacement would be provided between the housing and the rear assembly, the probability for kinking of the covering sleeve during its deployment around the insertion tube would be reduced.

In accordance with the invention the distribution bushing is provided with slots, which are cut in its cylindrical wall. Two such slots 116, 118 are seen in FIG. 8. The amount, width, length and configuration of the slots is selected in such a manner that the bushing 84 becomes elastically deformable when the rear assembly is tightly screwed on the sleeve anchoring member and the shelf region 102 presses on the proximal sealing element 82. By virtue of this provision the bushing length can be slightly shortened when the main body portion is screwed on the sleeve-anchoring member. This means that less strict dimensional tolerances should be met during designing and manufacturing of the bushing, the conical ring and the main body portion. This is advantageous, since it renders the manufacturing process less expensive and more convenient.

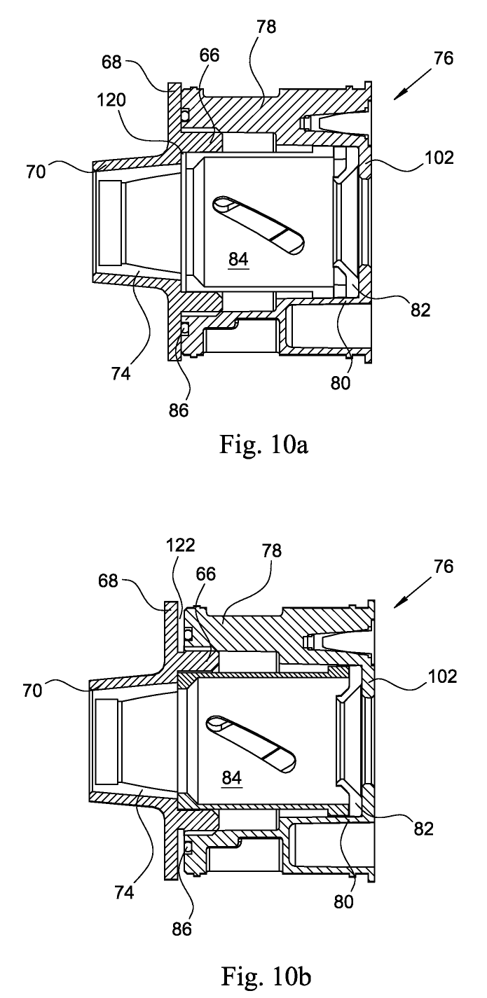

The problem associated with incompliance with the dimensional tolerances is illustrated in FIG. 10a and FIG. 10b.

FIG. 10a shows the situation when the bushing has been inadvertently manufactured with the length being shorter than required. It is seen that during assembling of the dispenser, there is a possibility that a gap 120 would be left. The gap 120 is formed between the flange portion 68 of the sleeve anchoring member 64 and the frontal end of the distribution bushing 84. Due to this gap it will not be possible to secure tightly the conical ring 74 in the skirt portion 70 and accordingly the proximal end of the sleeve would not be reliably anchored therebetween and there would be no reliable sealing between the proximal sealing element 82 and shelf region 102 of the shoulder 80.

FIG. 10b shows the situation when the bushing is longer than required. In this situation a gap 122 is formed between the flange portion 68 of the sleeve-anchoring member 64 and the frontal end of the main body portion 78. This gap prevents the distal sealing element 86 from sealing.

If the distribution bushing is elastically deformable, the gaps could be eliminated providing the bushing is manufactured with the length that is deliberately selected to slightly exceed the nominal length.

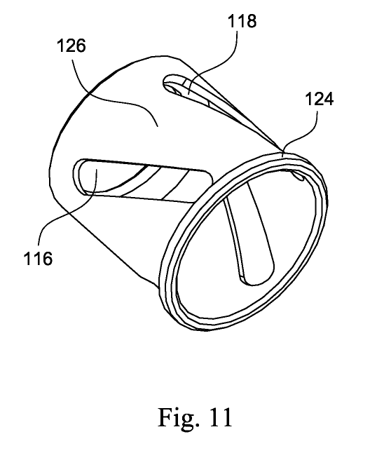

In FIG. 11 is shown an embodiment of the elastically deformable distribution bushing. The bushing is configured as a cylindrical tube having a short, rear portion 124 and a long frontal portion 126. The short portion has thickness of about 2 mm and the long portion has thickness of about 1 mm. The short portion is in contact with the proximal sealing element and the long portion is in contact with the proximal end of the conical ring and of the sleeve-anchoring member. A plurality of slots is cut in the cylindrical wall of the bushing. For the sake of brevity only two slots are designated by respective numerals 116, 118. In practice the bushing is made of a plastic material, e.g. PVC. Preferably it has six helicoidally directed slots, which have width of about 3 mm and length of about 15 mm. By virtue of such slots, it is possible to shorten the length of the bushing by 0.1-0.5 mm when is being screwed clockwise. The slots are evenly distributed and extend along the long portion and partially along the short portion.

The rear part of the bushing has increased thickness to impart some rigidity as might be required for reliable contact between the proximal sealing element and the bushing and thus to ensure proper sealing.

It can be appreciated that the length of the elastically deformable bushing can exceed the nominal size by 0.1-0.5 mm, which means that less strict dimensional tolerances have to be met.

It should be also appreciated that the lubricant can easy pass through the slots inside the bushing or be directly absorbed by the spongy liner protruding in the slots.

It should be appreciated that the present invention is not limited to the above-described embodiments and that changes and one ordinarily skilled in the art can make modifications without deviation from the scope of the invention, as will be defined in the appended claims.

For example instead of providing elastic deformation to the bushing by cutting helicoidally directed slots one could use slots having other configuration.

Furthermore, instead of using an elastically deformable bushing one could use a spring element deployed in the rear retaining assembly.

When used in the following claims, the meaning of terms “comprise”, “include”, “have” and their conjugates is “including but not limited to”.

It should also be appreciated that the features disclosed in the foregoing description, and/or in the following claims, and/or in the accompanying drawings may, both separately and in any combination thereof, be material for realizing the present invention in diverse forms thereof.

We claim:

1. A dispenser for storing of a disposable protecting sleeve suitable for covering of at least a portion of a flexible insertion tube of an endoscope, said dispenser comprising a frontal housing detachably connectable to a rear housing, wherein said frontal housing comprises a cover assembly detachably connected thereto and said rear housing comprises a sleeve anchoring member and a retaining assembly, said retaining assembly being provided with a distribution bushing through which a lubricant can be distributed and dislodged on the insertion tube, wherein said distribution bushing is elastically deformable.

2. The dispenser as defined in claim 1, wherein said frontal housing is detachably connected to the rear housing by a bayonet connection.

3. The dispenser as defined in claim 1, wherein said frontal housing is provided with a shield means.

4. The dispenser as defined in claim 1, wherein said shield means is provided with at least two lateral portions.

5. The dispenser as defined in claim 1, wherein said sleeve anchoring member and said retaining assembly are deployed within the rear housing.

6. The dispenser as defined in claim 1, further comprising a retainer bushing, said retainer bushing extending along the frontal and the rear housing such that room is provided for storing an auxiliary protecting sleeve.

7. The dispenser as defined in claim 1, wherein the sleeve anchoring member is provided with a conical skirt portion, with a flange portion and with a shoulder portion, wherein the dispenser comprises a conical ring, which is configured and dimensioned to enable a tight seat on the skirt portion.

8. The dispenser as defined in claim 7, wherein said retaining assembly being provided with a main body portion accommodating therein the distribution bushing and said main body portion has a shoulder provided with a shelf portion for abutting the distribution bushing.

9. The dispenser as defined in claim 8, wherein said distribution bushing being deployed within the main body portion for relative rotation with respect to the main body portion.

10. The dispenser as defined in claim 7, wherein said main body portion and the shoulder portion being provided with a thread such that said main body portion is screwable on the shoulder portion.

11. The dispenser as defined in claim 10, wherein a distal sealing element is provided between the flange portion of the sleeve anchoring member and the main body portion of the retaining assembly and wherein a proximal sealing element is provided between the distribution bushing and the shelf portion.

12. The dispenser as defined in claim 1, wherein a recess is provided in the rear part of the retaining assembly and a check valve is deployed in the recess, said check valve being accessible through the recess.

13. The dispenser as defined in claim 1, wherein said distribution bushing is provided with a spongy liner deployed on an inwardly facing surface of the distribution bushing.

14. The dispenser as defined in claim 1, wherein said distribution bushing is made of a plastic material.

15. The dispenser as defined in claim 14, wherein said distribution bushing is provided with a cylindrical wall and slots are cut in the cylindrical wall of the distribution bushing.
